SEQUENCE LISTING (3 sequences)

SEQ ID NO: 1 - DNA, 20 bp, synthetic construct
  source 1..20: mol_type = other DNA
agagtttgatcmtggctcag

SEQ ID NO: 2 - DNA, 19 bp, synthetic construct
  source 1..19: mol_type = other DNA
ggttaccttgttacgactt

SEQ ID NO: 3 - DNA, 1474 bp, Lactobacillus plantarum
  source 1..1474: mol_type = genomic DNA
ggcagtgcggcgtgctatacatgcagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggccaaaactcctaccggggaggcagcagtagggaatcttccaccaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaaccctttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaagtgacagatgg

(19) United States
(12) Patent Application Publication
(10) Pub. No.: US 2025/0027119 A1
(43) Pub. Date: Jan. 23, 2025

(54) LOCTOBACILLUS PLANTARUM 121-5 AND APPLICATION THEREOF IN PREPARING RESVERATROL BY CONVERTING POLYDATIN

(71) Applicant: SHAANXI INSTITUTE OF MICROBIOLOGY, XI’AN (CN)

(72) Inventors: WENJIAO XUE, XI’AN (CN); CHEN LIU, XI’AN (CN); CHAO AN, XI’AN (CN); JINGJING ZHANG, XI’AN (CN); YAO LIU, XI’AN (CN); HAO DING, XI’AN (CN); QIWEN ZHANG, XI’AN (CN); SAIJIAN MA, XI’AN (CN)

(21) Appl. No.: 18/776,658

(22) Filed: Jul. 18, 2024

(30) Foreign Application Priority Data
Jul. 18, 2023 (CN) 202310879961.3

(51) Int. Cl. C12P 7/22; C12N 1/20; C12N 9/42; C12R 1/25

(52) U.S. Cl. CPC C12P 7/22; C12N 1/20; C12N 9/2445; C12N 2500/16; C12N 2500/46; C12N 2500/50; C12N 2500/74; C12N 2500/84; C12R 2001/25

(57) ABSTRACT

A Lactobacillus plantarum 121-5 is disclosed. The preservation number is CGMCC No. 27418, and the Lactobacillus plantarum is preserved in the China general microbiological culture collection center on May 24, 2023. The Lactobacillus plantarum 121-5 has high beta-glucosidase activity, can efficiently convert polydatin to prepare resveratrol, and has short conversion time and high conversion rate. Meanwhile, the conversion condition is mild, safe and reliable, and the large-scale preparation is easy. The strain is directly applied to fermentation of foods and medicines (edible and medicinal plants or fruits and vegetables) containing polydatin, so that the resveratrol content in a fermentation product is improved, and the biological activity of the product is improved.

### SEQUENCE LISTING

The substitute sequence listing is submitted as a XML file filed via EFS-Web, with a file name of “Sequence Listing.xml”, a creation date of Jul. 17, 2024, and a size of 5,233 bytes. The substitute sequence Listing filed via EFS-Web is a part of the specification and is incorporated in its entirety by reference herein.

### TECHNICAL FIELD

The disclosure relates to the technical field of microorganisms, in particular to Lactobacillus plantarum 121-5 and application thereof in preparing resveratrol by efficiently converting polydatin.

### BACKGROUND ART

As a natural active polyphenol substance, the resveratrol has the effects of inhibiting tumor, resisting oxidation, resisting free radicals, resisting thrombus, resisting atherosclerosis, preventing and treating coronary heart disease, ischemic heart disease, hyperlipidemia and the like, and been listed as one of the most promising medicaments for resisting cardiovascular and cancer. Resveratrol also have multiple effects in skin care products, including antioxidant, antiaging, ultraviolet resistant, whitening and moisturizing effects, and thus has unique effects on skin aging (wrinkles and pigmentation) caused by various factors. Therefore, the resveratrol has wide applications in the market, and the production and preparation of the resveratrol poses economic and social benefits.

However, natural resveratrol takes little percentages in the world, and is mostly present in the plant body in the form of glycoside, i.e. the polydatin. The polydatin content in Polygonum cuspidatum, for embodiment, can be up to 2%, while the resveratrol content is only 0.1%-0.2%. The chemical synthesis cost for the resveratrol is high, so that polydatin is commonly used in industry as a raw material, and resveratrol is produced by a chemical catalysis method or a bioconversion method. The chemical catalysis method mainly adopts inorganic acid such as hydrochloric acid, sulfuric acid and the like as a catalyst to hydrolyze polydatin to prepare resveratrol. The acid catalytic hydrolysis method has higher hydrolysis temperature, the acid adopted has serious corrosion to equipment, and a large amount of acid wastewater is generated, polluting the environment. The bioconversion method mainly uses beta-glucosidase or microbe with beta-glucosidase activity as catalyst to hydrolyze polydatin to prepare resveratrol. In the US201110128953.2, a strain of giant knotweed rhizome endophyte Penicillium oxalicum J1 for converting polydatin into resveratrol ahs been disclosed. In the US201110130127.1, a strain of giant knotweed rhizome endophyte Penicillium oxalicum G3 for efficiently converting polydatin into resveratrol has been disclosed. In the US201410037538.X, a strain of beta-glucosidase high-yield bacterium Aspergillus oryzae and a method for preparing resveratrol by utilizing the strain to convert have been disclosed. In the US201611156766.4, a strain of resveratrol fermenting bacteria Bacillus safoci have been disclosed. However, the two species of bacteria are not in GRAS certification lists and have not been recognized as safe, such strains cannot be directly applied to the production of foods.

Therefore, the problem to be solved by the person skilled in the art is to discover the functional bacteria with safety performance for converting polydatin into resveratrol.

### SUMMARY

In view of the above, a Lactobacillus plantarum 121-5 has been disclosed, which has the capability of efficiently converting polydatin into resveratrol, and the conversion rate is 99%. Lactic acid bacteria are common probiotics, so that the lactic acid bacteria are high in safety and can be directly eaten, and the enzyme produced by the Lactobacillus plantarum 121-5 has high catalytic activity, selectivity and stability.

Following schemes in the disclosure have been adopted in order to achieve the above purpose.

The Lactobacillus plantarum 121-5 is classified and named as Lactobacillus plantarum, the preservation number is CGMCC No. 27418, and the Lactobacillus plantarum is preserved in the China general microbiological culture Collection center of the China Committee for culture Collection of microorganisms on May 24, 2023, and the preservation address is No. 1, Beichen west road, Chaoyang region of Beijing.

Another object of the present disclosure is to provide the application of Lactobacillus plantarum 121-5 in degrading glycosides.

Another object of the present disclosure is to provide the application of the above Lactobacillus plantarum 121-5 in industries that utilize beta-glucosidase.

The disclosure also aims to provide an application of the Lactobacillus plantarum 121-5 in preparing resveratrol by converting polydatin.

The disclosure also aims to provide a method for preparing resveratrol by utilizing the Lactobacillus plantarum 121-5 to transform polydatin, which includes the steps of dissolving polydatin in dimethyl sulfoxide, adding the polydatin into an MRS sugar-free culture medium, inoculating Lactobacillus plantarum 121-5 seed liquid according to 10% (v/v) of inoculation amount, and standing, fermenting and transforming for 4-16 hours under 37° C.

As a preferable technical scheme, the MRS sugar-free culture medium consists of 10.0 g of peptone, 10.0 g of beef extract, 5.0 g of yeast extract, 5.0 g of sodium acetate, 2.0 g of diamine citrate, 1 mL of TWEEN 80, 2.6 g of dipotassium hydrogen phosphate, 0.58 g of magnesium sulfate heptahydrate, 0.19 g of manganese sulfate heptahydrate, 1.0 L of distilled water and the pH is 6.3.

As a preferable technical scheme, the preparation method of the Lactobacillus plantarum 121-5 seed solution includes the steps of continuously activating Lactobacillus plantarum 121-5 preserved at the temperature of minus 80° C. for two generations, concentrating in an MRS liquid culture medium, and standing and culturing at the temperature of 37° C. for 16-30 hours.

The Lactobacillus plantarum 121-5 has high beta-glucosidase activity, can efficiently convert polydatin to prepare the resveratrol, and has short conversion time and high conversion rate. Meanwhile, the conversion condition is mild, safe and reliable, and the large-scale preparation is easy. The strain is directly applied to fermentation of foods and medicines (edible and medicinal plants or fruits and vegetables) containing polydatin, so that the resveratrol content in a fermentation product is improved, and the biological activity of the product is improved. The disclosure omits the steps of extracting the catalytic enzyme from the thalli and protecting the enzyme activity, and has simpler preparation, lower production cost and lower environmental pressure.

### DESCRIPTION OF THE ACCOMPANYING DRAWINGS

In order to provide a clearer explanation of the embodiments of the present disclosure or the technical solutions in the prior art, a brief introduction will be made to the accompanying drawings in the embodiments or the prior arts. Obviously, the accompanying drawings in the following description are only embodiments of the present disclosure. For those skilled in the art, other accompanying drawings can be obtained based on the provided drawings without creative efforts.

FIG. 1 shows the colony morphology of strain 121-5;

FIG. 2 shows the Gram staining microscopic examination of strain 121-5;

FIG. 3 shows the phylogenetic tree of strain 121-5;

FIG. 4 shows the color change results of the suspension/supernatant of strain 121-5 after reacting with pNPG;

FIG. 5 shows the standard curve of pNP;

FIG. 6 shows the release of pNP from bacterial suspension/supernatant of strain 121-5 after reacting with pNPG (μM);

FIG. 7 shows the HPLC plot of strain 121-5 converting Polygonatum cuspidatum glycoside to resveratrol.

### DETAILED DESCRIPTION OF THE EMBODIMENTS

The following description of the embodiments of the present disclosure will be made clearly and completely with reference to the accompanying drawings. The embodiments described hereinbelow are only some embodiments of the present disclosure, but not all embodiments. All other embodiments, which can be made by those skilled in the art based on the embodiments of the disclosure without making any creative efforts, are intended to be within the scope of the disclosure.

A Lactobacillus plantarum 121-5 and it's application are disclosed in the embodiments. Raw materials and reagents 121-5 used in the disclosure are commercially available, sources of the raw materials and the reagents are not particularly limited, and related methods, without special mention, are conventional methods and are not repeated herein.

### Embodiment I, Isolation and Identification of the Strain 121-5

(1) Separating and purifying strains: taking 0.5 g of manually fermented vegetables in the xi′ an region of Shaanxi, cutting the sample into small pieces of 0.3×0.3 cm with sterile scissors, adding 10 mL of sterile physiological saline, shaking for 3 min under vortex, and diluting to 10⁻⁷, respectively absorb 100 μL of 10⁻⁴˜10⁻⁷ diluent and coated on an MRS solid culture medium added with 2% calcium carbonate, standing culturing for 48 hours under 37° C., colonies with calcium dissolving rings are selected, streak purification is carried out on the MRS solid culture medium, and single colonies are selected for gram staining; selecting gram-positive bacterial strain with rod-shaped microscopic morphology, 25% glycerol, and freezing at −80° C. for later use.

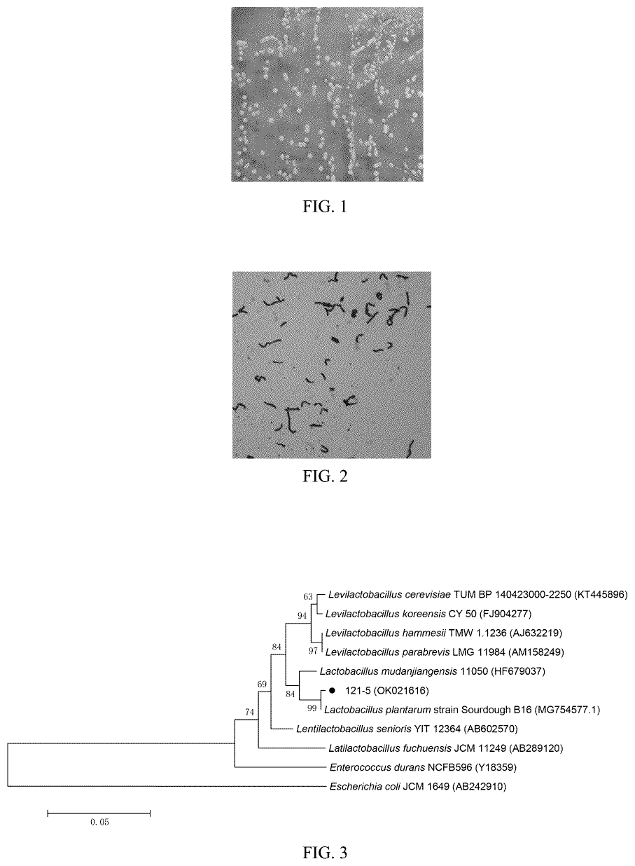

Bacterial colony of strain 121-5 is moist and has a white color shape, the surface of the bacterial colony is smooth and slightly convex (see FIG. 1), gram staining of the bacterial colony is positive (purple), and the bacterial colony is rod-shaped (see FIG. 2).
(2) Identification of strains: DNA of strain 121-5 was extracted using bacterial genome extraction kit (TaKaRa MiniBEST Bacterial Genomic DNA Extraction kit ver 2.0) and PCR amplification was performed using bacterial 16S rDNA universal primers: upstream primer 27F: AGAG TTTG ATCM TGGC TCAG, SEQ IN NO: 1; downstream primer 1492R: GGTT ACCT TGTT ACGA CTT, SEQ IN NO: 2. PCR amplification reaction System (50 μL): 5.0 μL of 10×Taq enzyme buffer, 0.4 μmol/L primer, 4 μL of 200 mmol/L dNTP, 40 ng of DNA template, 1U of Taq enzyme (TaKaRa), 34 μL of dd H₂O.

The PCR reaction was amplified as follows: pre-denaturation at 95° C. for 5 min; denaturation at 95° C. for 30 s, annealing at 55° C. for 1 min, extension at 72° C. for 1 min, 35 cycles were performed; finally, the extension is carried out for 5 min at 72° C. Reactions were performed on a Bio-Rad PCR instrument and the 16S rDNA amplified fragments were detected by 1% agarose gel electrophoresis and submitted to Shenzhen HuaDa Gene technologies Co. The sequencing result is compared with sequences in GenBank by Blast, the result shows that the gene sequence of the strain 16S rDNA (shown as SEQ ID No. 3) has the highest similarity with the gene sequence of Lactobacillus plantarum Sourdough B16 (MG 754577), a phylogenetic tree (see FIG. 3) is constructed by using MEGA-X software, the strain is known to be Lactobacillus plantarum (Lactobacillus plantarum) and is preserved in the China general microbiological culture Collection center of the China general microbiological culture Collection center for 24 months in 2023, the preservation address is No. 3, Beichen West road No. 1, chaoyang district, Beijing and the preservation number is: CGMCC No. 27418; taxonomies were named as Lactobacillus plantarum.

### Embodiment 2 Strain 121-5 Beta-Glucosidase Activity and Enzyme Localization

Continuously activating strain 121-5 frozen at −80° C. for two generations, inoculating into MRS liquid culture medium with 1% (v/v) inoculum size, standing at 37° C. for 18-24 hr to obtain fermentation liquor, centrifuging for 10 min (8000 r/min, 4° C.) to obtain supernatant and thallus, respectively, washing the thallus with CPB buffer (pH=6.0) once, suspending in CPB (pH 6.0), and regulating thallus concentration to 10⁸ cfu/mL. To ninety-six well plates were added 30 μL of bacterial suspension (IC) (or supernatant CFS), 20 μL of CPB buffer (pH 6.0) and 100 μL of pNPG in sequence, and incubated at 37° C. for 30 min. The reaction was terminated by immediately adding 50 μL of 1 mol/L sodium carbonate solution.

The above-mentioned measuring method is pNPG method, which is an indirect method for measuring beta-glucosidase enzyme activity, and its basic principle is to measure the concentration change of the product produced by enzyme reaction. The pNPG is a composition that can be hydrolyzed by glucosidase to pNP and glucose. Wherein the pNP has a yellow color and the enzyme activity can be determined by colorimetry.

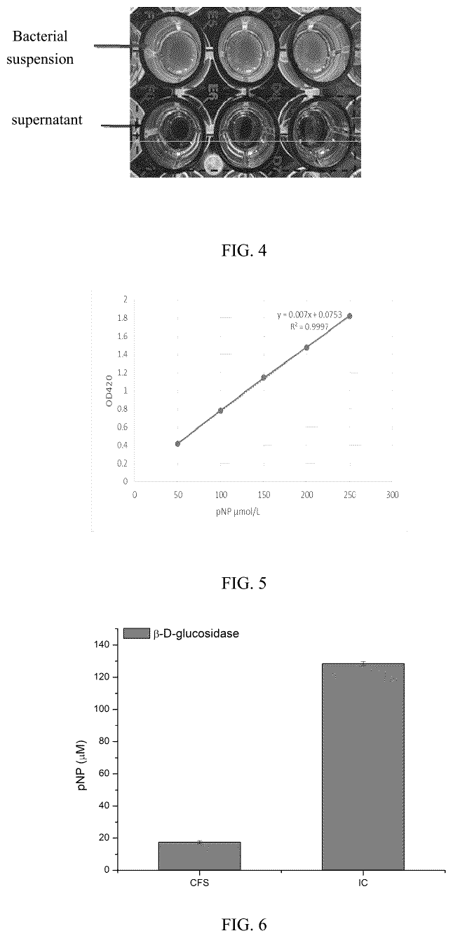

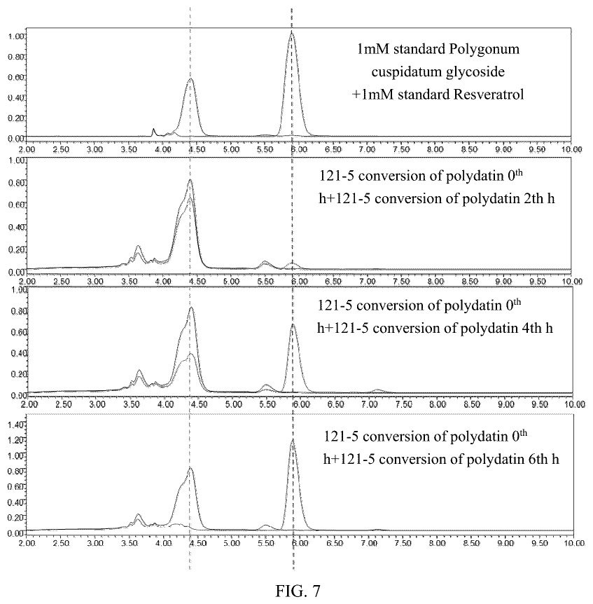

The observation result shows that the strain 121-5 bacterial suspension is yellow, has beta-glucosidase activity, the fermentation supernatant is colorless, and has no obvious beta-glucosidase activity (see FIG. 4), thus indicating that the strain 121-5 beta-glucosidase is intracellular enzyme. Further using an enzyme-labeled instrument to determine OD 420 nm. The absorbance was plotted as a pNP standard curve (see FIG. 5), and the pNP concentration after the reaction was calculated from the standard curve, which revealed that strain 121-5 had a high beta-glucosidase activity (see FIG. 6).

### Embodiment 3 Preparation of Resveratrol by Fermentation of Strain 121-5 to Convert Polydatin

(1) Seed liquid preparation: the strain 121-5 is frozen and preserved at the temperature of −80° C., continuously activating the strain for two generations and inoculated into the MRS liquid culture medium, and is subjected to stationary culture at the temperature of 37° C. for 22 hours, so as to obtain seed liquid.

The MRS culture medium includes the following components: 10.0 g of peptone, 10.0 g of beef extract, 5.0 g of yeast extract, 20.0 g of glucose, 5.0 g of sodium acetate, 2.0 g of diamine citrate, 1 mL of TWEEN 80, 2.6 g of dipotassium hydrogen phosphate, 0.58 g of magnesium sulfate heptahydrate, 0.19 g of manganese sulfate heptahydrate, 1.0 L of distilled water and the pH of the medium is 6.3. The MRS medium was sterilized at 121° C. for 20 min.
(2) Fermentation and conversion: 200 mM polydatin mother liquor (DMSO dissolving) is prepared, and then mixed with MRS sugar-free culture medium (without glucose, and the rest is the same as the MRS culture medium in the step 1) to prepare the polydatin conversion solution with the final concentration of 1 mM. Taking 10 mL of the strain 121-5 obtained in the step (1), centrifuging at 4° C. at 800 rpm for 10 min to remove supernatant, washing the thalli twice with PBS, re-suspending the thalli by 1 mL of PBS, and inoculating into 100 mL of a polydatin conversion solution with the final concentration of 1 mM, standing at 37° C. for fermentation and conversion for 6 h.
(3) HPLC detection: sampling fermentation liquid in the 0th, the 2th, the 4th and the 6 th hour(s) of fermentation and conversion in the step (2), freeze-drying, adding DMSO for full dissolution, filtering with a 0.45 μm filter membrane, fixing the volume, and measuring the content of polydatin and resveratrol by utilizing HPLC.

The HPLC detection conditions were: the column was C18 strain (150 mm×4.6 mm, 5 μm), mobile phase methanol:water (60:40, v/v), flow rate 0.8 mL/min, column temperature 30° C., detection wavelength 279 nm.

HPLC detection results of the process of converting polydatin into resveratrol by the strain 121-5 are shown in FIG. 5. 1 mM polydatin was completely converted at 6 hours of conversion, and the conversion rate of resveratrol was calculated to be 99%.

In the present specification, each embodiment is described in a progressive manner, and each embodiment is mainly described in a different point from other embodiments, and identical and similar parts between the embodiments are all enough to refer to each other.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

What is claimed is:

1. A Lactobacillus plantarum 121-5 with a preservation number of CGMCC No. 27418, and the Lactobacillus plantarum 121-5 is preserved in the China general microbiological culture Collection center of China Committee for culture Collection of microorganisms.

2. An application of the Lactobacillus plantarum 121-5 of claim 1 for degrading glycosides.3.

3. Application of the Lactobacillus plantarum 121-5 of claim 1 utilizing beta-glucosidase activity.

4. Application of the Lactobacillus plantarum 121-5 of claim 1 for the conversion of polydatin to resveratrol.

5. A method for preparing resveratrol by using the Lactobacillus plantarum 121-5 of claim 1 to transform polydatin, comprising
dissolving polydatin is in dimethyl sulfoxide,
adding into an MRS sugar-free culture medium,
inoculating with Lactobacillus plantarum 121-5 seed solution according to 10 percent of inoculation amount, and
subjecting to standing fermentation and transformation at 37° C. for 4-16 hours.

6. The method of claim 5, wherein the MRS sugarless medium comprises:
peptone 10.0 g, beef extract 10.0 g, yeast extract 5.0 g, sodium acetate 5.0 g, diamine citrate 2.0 g, 1 ml TWEEN 80, dipotassium hydrogen phosphate 2.6 g, magnesium sulfate heptahydrate 0.58 g, manganese sulfate heptahydrate 0.19 g, distilled water 1.0 L;
wherein a pH of the medium is 6.3.

7. The method of claim 5, wherein the Lactobacillus plantarum 121-5 seed solution is prepared by continuously activating Lactobacillus plantarum 121-5 preserved at −80° C. for two generations, concentrating in MRS liquid culture medium, and standing at 37° C. for 16-30 hours.
